(19) United States
(12) Patent Application Publication
(10) Pub. No.: US 2023/0086522 A1
(43) Pub. Date: Mar. 23, 2023

(54) METHODS FOR DEHYDRATING AND REHYDRATING MYCELIUM

(71) Applicant: EMERGY INC., Boulder, CO (US)

(72) Inventors: Justin WHITELEY, Lafayette, CO (US); Mitchell Tyler HUGGINS, Boulder, CO (US); Behroze Mistry, Boulder, CO (US); Debbie Downing, Westminster, CO (US)

(73) Assignee: EMERGY INC., Boulder, CO (US)

(21) Appl. No.: 17/904,215

(22) PCT Filed: Feb. 10, 2021
(86) PCT No.: PCT/US2021/017493

§ 371 Date: Aug. 12, 2022

Related U.S. Application Data
(60) Provisional application No. 62/976,939, filed on Feb. 14, 2020.

(51) Int. Cl. A23J 1/00; A23J 3/22; A23J 3/20; A23L 31/00; A23L 5/00; C12N 1/14

(52) U.S. Cl. CPC A23J 1/008; A23J 3/227; A23J 3/20; A23L 31/00; A23L 5/55; C12N 1/14; A23V 2002/00

(57) ABSTRACT

A method of dehydrating and rehydrating mycelium includes growing fungal cells in a growth media such that the fungal cells produce a mycelium mass having a protein content of greater than 40 wt % of a dry mass of the mycelium mass. The method includes separating the mycelium mass from the growth media, compacting the mycelium mass, and dehydrating the compacted mycelium mass to produce a dehydrated mycelium mass having a moisture content in a range of 5 wt % to 60 wt % and a first hardness in a range of 0.007 kgf/mm² to 0.018 kgf/mm². The method includes rehydrating the dehydrated mycelium mass to form a rehydrated mycelium mass having a moisture content of greater than 60 wt % and a second hardness in a range of 0.00035 kgf/mm² to 0.007 kgf/mm².

### CROSS-REFERENCE TO RELATED PATENT APPLICATIONS

The present application claims the benefit of and priority to U.S. Provisional Patent Application No. 62/976,939 filed Feb. 14, 2020, the entire disclosure of which is incorporated by reference herein.

### TECHNICAL FIELD

The present disclosure relates generally to the field of fungal mycelium based edible meat substitute products.

### BACKGROUND

Demand for edible products that can provide a high protein content which is drawn from a non-animal source is increasing. Driven by increasing awareness of personal health, edible products that include non-animal sourced components such as proteins and fibers are considered as a healthier alternative to animal protein based products. In particular, there is growing demand for edible meat substitutes that mimic meat in its composition and texture but are composed of non-animal components, which can reduce reliance on animals such as cows, chickens, and pigs, and reduce the carbon footprint posed by such animals. Thus, there is a need for non-animal protein sources that can facilitate large scale production and adoption of non-animal based edible products.

### SUMMARY

Embodiments described herein relate generally to methods for dehydrating and rehydrating mycelium for obtaining edible meat substitute products that resemble animal meat in their texture and morphology.

In some embodiments, a method comprises growing fungal cells in a growth media such that the fungal cells produce a mycelium mass having a protein content of greater than 40 wt % of a dry mass of the mycelium mass; separating the mycelium mass from the growth media; compacting the mycelium mass to produce a compacted mycelium mass having a moisture content in a range of 65 wt % to 85 wt %; dehydrating the compacted mycelium mass to produce a dehydrated mycelium mass having a moisture content in a range of 5 wt % to 60 wt % and a first hardness in a range of 0.007 kgf/mm² to 0.018 kgf/mm²; and rehydrating the dehydrated mycelium mass to form a rehydrated mycelium mass having a moisture content of greater than 60 wt % and a second hardness in a range of 0.00035 kgf/mm² to 0.007 kgf/mm².

In some embodiments, a method comprises growing fungal cells in a growth media such that the fungal cells produce a mycelium mass having a protein content of greater than 40 wt % of a dry mass of the mycelium mass; separating the mycelium mass from the growth media; compacting the mycelium mass to produce a compacted mycelium mass having a moisture content in a range of 65 wt % to 85 wt %; dehydrating the compacted mycelium mass at a temperature in a range of 30° C. to 60° C. and a relative humidity in a range of 30% to 50% for a time period to produce a dehydrated mycelium mass having a moisture content of less than 20 wt %, the dehydrated mycelium mass having a volume that is less than 50% of the compacted mycelium mass.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several implementations in accordance with the disclosure and are therefore, not to be considered limiting of its scope, the disclosure will be described with additional specificity and detail through use of the accompanying drawings.

FIG. 1 is a flow chart of an example method for dehydrating and rehydrating mycelium, according to an embodiment.

FIG. 2 is a flow chart of an example method for dehydrating and rehydrating mycelium, according to an embodiment.

FIG. 3 is a flow chart of an example method for dehydrating mycelium, according to an embodiment.

FIG. 4A illustrates a bar chart of hardnesses of a compacted mycelium mass, a dehydrated and rehydrated mycelium mass, a second dehydrated mycelium mass formed by dehydrating the rehydrated mycelium mass, raw chicken, and cooked chicken, according to an embodiment.

FIG. 4B illustrates a table of moisture contents of a compacted mycelium mass, a dehydrated and rehydrated mycelium mass, a second dehydrated mycelium mass formed by dehydrating the rehydrated mycelium mass, raw chicken, and cooked chicken, according to an embodiment.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

Embodiments described herein relate generally to methods for dehydrating and rehydrating mycelium for obtaining edible meat substitute products that resemble animal meat in their texture and morphology. Particularly, various embodiments described herein provide methods of growing fungal cells to produce a mycelium mass, separating the mycelium mass, compacting the mycelium mass to form a compacted mycelium mass, dehydrating the compacted mycelium mass, and rehydrating the dehydrated mycelium mass. Textural changes can be achieved by dehydrating and then rehydrating mycelium. The shear cutting, bending moment, and hardness of a mycelium mass that has undergone dehydration and dehydration can differ from those of a mycelium mass that has the same moisture content but has not undergone these processes. Various embodiments also relate to adding food additives to form an edible food product or edible meat substitute product. The edible meat substitute product can include a mycelium mass having a protein content of greater than 40 wt % of a dry mass of the mycelium mass.

Various embodiments of the methods of growing fungal mycelium and forming edible products therefrom may provide one or more benefits including, for example: (1) providing edible products that include protein from a non-animal source, i.e., fungal mycelium, thereby reducing dependence on animal sources of proteins and reducing their carbon footprint; and (2) providing edible meat substitute products that feel and taste like real meat while delivering a high protein content.

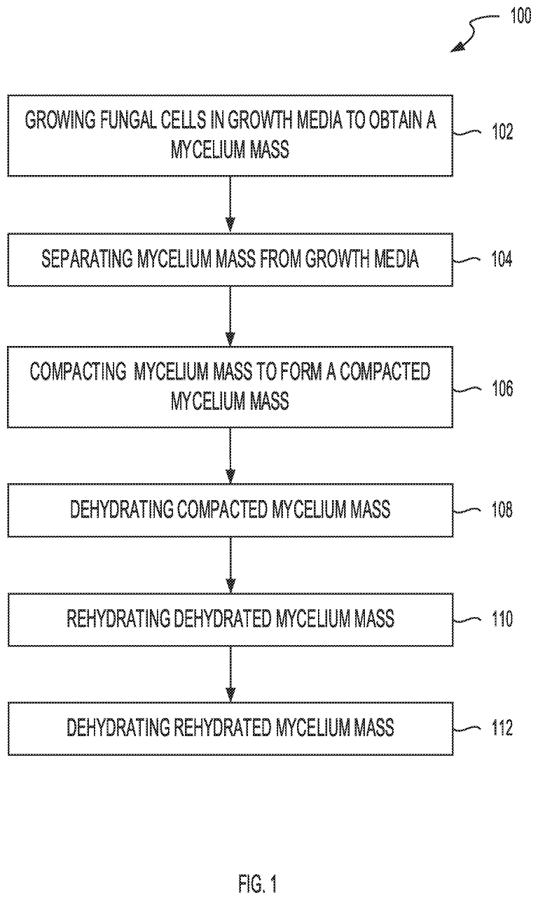

FIG. 1 illustrates a flow chart of an example method for forming an edible meat substitute product, according to an embodiment. In brief overview, the method 100 may include growing fungal cells in a growth media, at 102. The method 100 may include separating mycelium mass from the growth media, at 104. The method 100 may include compacting the mycelium mass, at 106. The method 100 may include dehydrating the compacted mycelium mass, at 108. The method 100 may include rehydrating the dehydrated mycelium mass, at 110. The method 100 may include dehydrating the rehydrated mycelium mass, at 112.

In further detail, the method 100 may include growing fungal cells in a growth media, at 102. The fungal cells can include fungi from Ascomycota and Zygomycota, including the genera Aspergillus, Fusarium, Neurospora, and Monascus. Other species include edible varieties of Basidiomycota and genera Lentinula. One genus is Neurospora, which is used in food production through solid fermentation. The genus of Neurospora are known for highly efficient biomass production as well as ability to break down complex carbohydrates. For certain species of Neurospora, no known allergies have been detected and no levels of mycotoxins are produced. In addition to monocultures of filamentous fungi, multiple strains can be cultivated at once to tune the protein, amino acid, mineral, texture, and flavor profiles of the final biomass.

The growth media may be contained in a vessel, such as a vat capable of growing several kilograms of the fungal mycelium. The growth media can be referred to as an original growth media. The method 100 may include growing fungal cells in a growth media such that the fungal cells produce mycelium. The growth media can include nutrients (e.g., sugar, nitrogen-containing compounds, or phosphate-containing compounds). The growth media can include one or more of a sugar, a nitrogen-containing compound, and a phosphate-containing compound. The sugar can be in the range of 5 g/L to 50 g/L. For example, the sugar can be 5 g/L, 10 g/L, 20 g/L, 30 g/L, 40 g/L, or 50 g/L, inclusive. The sugar can include sucrose, glucose, fructose, molasses, or a mixture of sugars. The nitrogen-containing compound can be in the range of 0.5 g/L to 10 g/L. For example, the nitrogen-containing compound can be 0.5 g/L, 1 g/L, 2 g/L, 3 g/L, 4 g/L, 5 g/L, or 10 g/L, inclusive. The nitrogen-containing compound can include ammonium hydroxide, ammonium nitrate, ammonium sulfate, ammonium chloride, urea, yeast extract, peptone, or a mixture of nitrogen-containing compounds. The phosphate-containing compound can be in the range of 0.1 g/L to 5 g/L. For example, the phosphate-containing compound can be 0.1 g/L, 0.2 g/L, 0.3 g/L, 0.4 g/L, 0.5 g/L, 1 g/L, 2 g/L, 3 g/L, 4 g/L, or 5 g/L, inclusive. The phosphate-containing compound can be potassium phosphate, sodium phosphate, phosphoric acid, or a mixture of phosphate-containing compounds.

The fungal cells can be grown at a temperature in a range of 25° C. to 40° C., inclusive. The fungal cells can be grown for a time period in a range of 12 hours to 48 hours, inclusive. Growing fungal cells can produce a yield of 5 g/L to 20 g/L of fungal cell dry weight. The mycelium can have a protein content of greater than 40 wt % (dry weight). In some embodiments, the mycelium may have a protein content of 50% to 65%, inclusive (dry weight). The mycelium can have a combined methionine and cysteine content of at least 25 mg/g crude protein.

In some embodiments, the method 100 may include removing a volume of a broth (e.g., siphoned broth). The siphoned broth can contain the fungal cells and the growth media. For example, the broth can include a solution containing the fungal cells and the growth media. Removing a volume of broth can include discretely removing a volume of broth. For example, a volume of broth can be siphoned from a container containing the broth in a batch process, or be continuously removed from the broth. For example, a volume of broth can flow out of the container containing the broth in a continuous process.

The method 100 may include adding fresh growth media to a container containing the broth. The broth can be a fermentation broth. Nutrients (e.g., sugar, phosphate-containing compound, or nitrogen-containing compound) can be added in a batch growth configuration. For example, the nutrients can be added after a predetermined amount of time (e.g., after 1 hour, 2 hours, 3 hours, 6 hours, or 12 hours, inclusive). The concentrations of none or at least one of the nutrients of the fresh growth media can be brought to the concentrations of nutrients of the original growth media described in operation 102. The fresh growth media can have a volume that is greater than, less than, or equal to a volume of growth media that was lost from the original growth media during growth of the fungal cells in the original growth media.

In one example, after 6 hours, the concentration of sugar, phosphate-containing compound, and nitrogen-containing compound in the fresh growth media is increased. Nutrients are added to the broth to create a new broth. Nutrients are added to the broth to bring the concentrations of sugar, phosphate-containing compound, and nitrogen-containing compound of the new broth to the concentrations of sugar, phosphate-containing compound, and nitrogen-containing compound, respectively of the original growth media.

In one example, after at least 12 hours, 50% to 95% of the broth can be removed. Fresh media can be added containing nutrients (e.g., sugar, phosphate-containing compound, or nitrogen-containing compound). The nutrient concentration of the broth can be increased by adding fresh growth media.

Nutrients can be added in a continuous growth configuration. For example, a volume of broth (e.g., 0.01 vol %, 1 vol %, 5 vol %, 10 vol %, 25 vol %, 50 vol %, or 95 vol %, inclusive) can be removed from the container containing the fungal cells and the growth media. Fresh growth media can be added to the container containing the broth. The fresh growth media can be provided as a continuous flow. The volume of the broth in the container can be monitored to stay at a specified level. For example, the volume of the broth in the container can stay at a fixed volume. The volume of fresh growth media that is added can be equal to the volume of broth that is lost from the container.

The method 100 may include growing fungal cells in a growth media such that the fungal cells produce a mycelium mass having a protein content of greater than 40 wt % of a dry mass of the mycelium mass. For example, the mycelium mass can have a protein content of 45 wt %, 50 wt %, 60 wt %, 70 wt %, 80 wt %, or 90 wt %, inclusive, of the dry mass of the mycelium mass.

The method 100 includes separating the mycelium mass from the growth media, at 104. Separating the mycelium mass from the growth media can be performed using gravity straining, centrifugation, a belt press, a filter press, a mechanical press, a drum dryer, or any other suitable process. The separated mycelium mass can have a moisture content of greater than 90 wt %. For example, the separated mycelium mass can have a moisture content of 91 wt %, 92 wt %, 93 wt %, 94 wt %, 95 wt %, 96 wt %, 97 wt %, 98 wt %, or 99 wt %, inclusive. During the separation process, the mycelium mass can be washed with water, ethanol, acid, base or other solvent. Recovered filtrate can be reused or discarded. Cell walls of the mycelium mass can be disrupted, for example, through lysing. Lysis may be performed by adjusting the pH to below 4 or above 9, by adding lysis enzymes, by raising the temperature in a range of 40° C. and 60° C., inclusive, in a range of 1 and 24 hours, inclusive, or any other suitable lysis method. Following separation, additives (e.g., food additives) can be mixed with the mycelium mass. Additives can include vegetable or animal proteins, fats, emulsifiers, thickeners, stabilizers, and flavoring, for example, when the mycelium mass is being formed into an edible product.

The method 100 may include compacting the mycelium mass to yield a compacted mycelium mass, at 106. Compacting the mycelium mass may include pressing the mycelium mass to decrease a volume of the mycelium mas and produce a compacted mycelium mass. The compacted mycelium mass can have a moisture content in a range of 65 wt % to 85 wt %. For example, the compacted mycelium mass can have a moisture content of 65 wt %, 70 wt %, 75 wt %, 80 wt %, or 85 wt %, inclusive.

The method 100 may include dehydrating the compacted mycelium mass to produce a dehydrated mycelium mass, at 108. Dehydrating the compacted mycelium mass may include decreasing the moisture content of the compacted mycelium mass to produce a dehydrated mycelium mass. The dehydrated mycelium mass can have a moisture content in a range of 5 wt % to 60 wt %. For example, the dehydrated mycelium mass can have a moisture content of 5 wt %, 10 wt %, 15 wt %, 20 wt %, 25 wt %, 30 wt %, 35 wt %, 40 wt %, 45 wt %, 50 wt %, 55 wt %, or 60 wt %, inclusive. The dehydrated mycelium mass can have a first hardness in a range of 0.007 kgf/mm² to 0.018 kgf/mm², inclusive. Dehydrating the compacted mycelium mass can include dewatering the mycelium mass. For example, the compacted mycelium mass can be thermally dried, for example, to a moisture content in a range of 5 wt % to 60 wt %, inclusive. The compacted mycelium mass can be thermally dried at a specified temperature (e.g., 30° C., 50° C., 75° C., or 90° C., inclusive). The compacted mycelium mass can be dehydrated at a temperature of less than 60° C. For example, the compacted mycelium mass can be dehydrated at a temperature of 55° C., 50° C., 45° C., 40° C., or 35° C., inclusive. The compacted mycelium mass can be dehydrated at a humidity of less than 30%. For example, the compacted mycelium mass can be dehydrated at a humidity of 25%, 20%, or 15%, inclusive. In some embodiments, the compacted mycelium mass can be thermally dried with forced air. In some embodiments, dewatering the compacted mycelium mass can include applying a mechanical force (e.g., via a press, a plunger, etc.) to remove water from the mycelium. Dehydrating (e.g., via thermal or mechanical drying) the compacted mycelium mass produces the dehydrated mycelium mass.

Hardness measurements are achieved by using a TA.XT Texture Analyzer using a rounded ball probe tip and pressing into a flat surface of the product in question. A 19 mm diameter tip was indented into the product until 40% compression was achieved. Hardness is then taken as the peak force divided by the cross sectional area of the probe tip.

The method 100 may include rehydrating the dehydrated mycelium mass to form a rehydrated mycelium mass, at 110. Rehydrating the dehydrated mycelium mass may include increasing the moisture content of the dehydrated mycelium mass to produce the rehydrated mycelium mass. The rehydrated mycelium mass can have a moisture content greater than 70 wt %. For example, the dehydrated mycelium mass can have a moisture content of 75 wt %, 80 wt %, 85 wt %, 90 wt %, or 95 wt %, inclusive. The rehydrated mycelium mass can have a second hardness in a range of 0.00035 kgf/mm² to 0.007 kgf/mm². The second hardness can be different from the first hardness and is similar to hardness of animal meat such as raw chicken. Therefore, by dehydrating and rehydrating the mycelium mass as described herein yields an edible meat substitute that looks and feels similar to animal meat.

The volume of the dehydrated mycelium mass can be equal to or greater than 75% of a volume of the compacted mycelium mass. For example, the volume of the dehydrated mycelium mass can be 75%, 80%, 85%, 90%, 95%, or 100%, inclusive, of the volume of the compacted mycelium mass.

Rehydrating the dehydrated mycelium mass can include soaking the dehydrated mycelium mass in an aqueous solution (e.g., a marinade) comprising a flavor, a food coloring, fats, spices, and/or additives. Any suitable food additive can be used such as, for example, vegetable or animal proteins, fats, emulsifiers, thickeners, stabilizers, flavoring, pH adjusters, oils, spices, salts, etc. For example, the flavor of the dehydrated mycelium mass can be enhanced by adding different oils. Non-limiting examples of oils include nut-derived, vegetable-derived, plant-derived, and animal-derived oils. Oils can be added to the fermentation media to have the multi-purpose of acting as an antifoaming agent, a carbon source for the fungus, and to integrate extra/intracellularly into the mycelium mass. Alternatively, oil can be integrated into the mycelium mass following harvesting or following cooking. The dehydrated mycelium mass can have food additives introduced by various operations. For example, the dehydrated mycelium mass can be soaked in additional ingredients. The dehydrated mycelium mass can be injected with additional ingredients. The dehydrated mycelium mass can be coated in additional ingredients. Additional ingredients can be dispersed into the dehydrated mycelium mass by applying additional pressure or vacuum conditions. Additional ingredients can include vegetable or animal proteins, fats, emulsifiers, thickeners, stabilizers, flavoring, pH adjusters, etc.

Rehydrating the dehydrated mycelium mass can include soaking the dehydrated mycelium mass for a time period in a marinade heated to a temperature sufficient to kill greater than 99% of live fungal cells present in the dehydrated mycelium mass. The dehydrated mycelium mass can be soaked in marinade heated to a temperature of 50° C. to 200° C. For example, the dehydrated mycelium mass can be soaked in marinade heated to a temperature of 50° C., 55° C., 60° C., 65° C., 70° C., 75° C., 80° C., 85° C., 90° C., 95° C., 100° C., or 200° C. inclusive.

The method 100 may include dehydrating the rehydrated mycelium mass, at 112. Dehydrating the rehydrated mycelium mass may include dehydrating the rehydrated mycelium mass to produce a second dehydrated mycelium mass. The second dehydrated mycelium mass can have a moisture content in a range of 45 wt % to 75 wt %. For example, the second dehydrated mycelium mass can have a moisture content of 45 wt %, 50 wt %, 55 wt %, 60 wt %, 65 wt %, 70 wt %, 75 wt %, inclusive. The second dehydrated mycelium mass can have a third hardness in a range of 0.0035 kgf/mm² to 0.014 kgf/mm². The rehydrated mycelium mass can be dehydrated at a temperature in a range of 40° C. to 100° C. For example, the rehydrated mycelium mass can be dehydrated at a temperature of 40° C., 45° C., 50° C., 55° C., 60° C., 65° C., 70° C., 75° C., 80° C., 85° C., 90° C., 95° C., or 100° C., inclusive. The rehydrated mycelium mass can be dehydrated at a relative humidity of less than 20%. For example, the rehydrated mycelium mass can be dehydrated at a relative humidity of 15%, 10%, or 5%, inclusive. The second dehydrated mycelium mass can have a hardness more similar to cooked animal meat such as chicken.

The second dehydrated mycelium mass can have a moisture content of less than 50 wt %. For example, the second dehydrated mycelium mass can have a moisture content of 20%, 25%, 30%, 35%, 40%, or 50%, inclusive. The second dehydrated mycelium mass can have a third hardness in a range of 0.011 kgf/mm² to 0.042 kgf/mm², inclusive. The rehydrated mycelium mass can be dehydrated at a temperature in a range of 30° C. to 100° C. For example, the rehydrated mycelium mass can be dehydrated at a temperature of 30° C., 40° C., 45° C., 50° C., 55° C., 60° C., 65° C., 70° C., 75° C., 80° C., 85° C., 90° C., 95° C., or 100° C., inclusive. The rehydrated mycelium mass can be dehydrated at a relative humidity of less than 20%. For example, the rehydrated mycelium mass can be dehydrated at a relative humidity of 15%, 10%, or 5%, inclusive.

Following are some examples of growing fungi and obtaining a mycelium mass having a protein content of greater than 40 wt %. These examples are for illustrative purposes only and should not be construed as limiting the disclosure in any shape or form.

In one example, Neurospora crassa (N. crassa) was grown in batch configuration in a 10 L benchtop reactor. N. crassa is first grown on agar slants and incubated for 3 days at 32° C. Conidia or spores of the N. crassa are transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 6.6 g/L ammonium sulfate, 2 g/L potassium phosphate monobasic, 1 g/L sodium citrate, 0.2 g/L magnesium sulfate, 0.1 g/L calcium chloride, and trace elements. Aeration is set at 1.5 vvm and agitation at 350 rpm. The pH is adjusted and held at 5.8 using a 6 N potassium hydroxide buffer. After 24 hours, the mycelium is harvested using a cheese cloth, pressed into a stainless steel rectangular mold, and completely dried in a dehydrator set at 74° C. The total cell dry weight is 9.5 g/L. Protein analysis yields a crude protein content of 57 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. Conidia or spores of the N. crassa are transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 6.6 g/L ammonium sulfate, 1 g/L potassium phosphate monobasic, 0.2 g/L magnesium sulfate, 0.1 g/L calcium chloride, and trace elements. Aeration is set at 1.5 vvm and agitation at 350 rpm. The pH is adjusted and held at 5.8 using a 6 N potassium hydroxide buffer. After 24 hours, the mycelium is harvested using a cheese cloth, pressed into a stainless steel rectangular mold, and completely dried in a dehydrator set at 74° C. The total cell dry weight is 9 g/L. Protein analysis yields a crude protein content of 55 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 30 g/L sucrose, 6.6 g/L ammonium sulfate, 1 g/L potassium phosphate monobasic, 0.2 g/L magnesium sulfate, 0.1 g/L calcium chloride, and trace elements. Aeration is set at 1.5 vvm and agitation at 350 rpm. The pH is adjusted and held at 5.8 using a 6 N potassium hydroxide buffer. After 24 hours, the mycelium is harvested using a cheese cloth, pressed into a stainless steel rectangular mold, and completely dried in a dehydrator set at 74° C. The total cell dry weight is 11 g/L. Protein analysis yields a crude protein content of 63 wt %. Amino acid analysis yields a PDCAAS score of 1.0 for the fibrous mycelium mass. The fibrous mycelium mass has a combined methionine and cysteine content of 27 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 3.25 g/L urea, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, pressed into a stainless steel rectangular mold, and completely dried in a dehydrator set at 74° C. The total cell dry weight is 8.5 g/L. Protein analysis yields a crude protein content of 56 wt %. Amino acid analysis yields a PDCAAS score of 1.0. The fibrous mycelium mass has a combined methionine and cysteine content of 25 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 15% ammonium hydroxide buffer. After 24 hours, the mycelium is harvested using a cheese cloth, dewatered in a cider press, and completely dried in a dehydrator set at 74° C. The total cell dry weight is 10 g/L. Protein analysis yields a crude protein content of 60 wt %. Amino acid analysis yields a PDCAAS score of 1.0. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 12 hours, 10 g/L sucrose and 1 g/L ammonium nitrate is added to the system. After 24 hours total, the mycelium is harvested using a cheese cloth, dewatered in a cider press, and completely dried in a dehydrator set at 74° C. The total cell dry weight is 12 g/L. Protein analysis yields a crude protein content of 60 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, 90% of the media is harvested; new media is added in the concentrations of above to bring the total system back to 10 L. The new sequential batch time is reduced to 12 hours. Every 12 hours 90% is harvested and the fed-batch process is repeated again. The process was carried out for 60 hours. The harvested cell dry weight is 9.5 g/L. Protein analysis yields a crude protein content of 60 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, 90% of the media is harvested; new media is added in the concentrations of above to bring the total system back to 10 L. The new sequential batch time is reduced to 12 hours. Every 12 hours 90% is harvested and the fed-batch process is repeated again. The process was carried out for 60 hours. Following straining with cheese cloth and pressing, all media is collected, autoclaved and reused by only adding 20 g/L sucrose, 2 g/L ammonium nitrate, and 1 g/L potassium phosphate monobasic. The repeated fed-batch process is carried out for 60 hours total. The harvested cell dry weight is 9.5 g/L. Protein analysis yields a crude protein content of 60 wt %. Amino acid analysis yields a PDCAAS score of 1.0 for the fibrous mycelium mass. The fibrous mycelium mass has a combined methionine and cysteine content of 26 mg/g crude protein.

A method of batch growth is described herein. Growth experiments were conducted in 1 L of fresh residual water. Batch cultures were incubated at 30° C. for 1-3 days (120 rpm) under constant light. Harvesting of biomass was performed using a vacuum filtration flask and then subsequently dried at 105° C.

The crude protein content of the filamentous fungus can be increased by supplementing with additional nitrogen sources. Non-limiting examples include supplementing with gaseous ammonia, liquid ammonia, ammonium nitrate, ammonium sulfate, sodium nitrate, yeast extract, urea, peptone, or other organic nitrogen source. A nitrogen source can be added with other pH buffering components. Non-limiting examples include acids, phosphates, borates, sulfates, and bases.

Following are some examples of growing fungi and dehydrating and rehydrating a mycelium mass. These examples are for illustrative purposes only and should not be construed as limiting the disclosure in any shape or form.

In one example, Neurospora crassa (N. crassa) was grown in a 200 L stirred tank bioreactor under pre-set conditions. Following full growth, N. crassa biomass (mycelium) was collected through a 200 PEM nylon mesh bag. The moisture content of the mycelium at this stage was approximately 95%. The high moisture mycelium is added to a perforated, stainless-steel mold consisting of base and follower. A pressure of 100 psi is applied to the mold lid compacting the mycelium into a rigid block of approximately 75% moisture content. The block is then sliced or broken into pieces of desired size and shape. Molded mycelium slices are then placed onto a perforated tray and placed in a dehydrator. The dehydration parameters are set to a temperature of 60° C. and relative humidity of less than 20%. The molded mycelium slices dehydrate to less than 20% moisture content in under 6 hours. The final volume of the dehydrated mycelium is greater than 75% of the initial pressed mycelium.

The dehydrated mycelium can then be rehydrated with flavor, color, fats, or other functional food ingredients to get back to the original 75% moisture content or greater. Because of the specific dehydration parameters and low volume reduction, the rehydration ratio is quite high. At this point, the rehydrated mycelium is far less rigid than the original pressed mycelium and has the texture of animal meat such as chicken.

In another example, dehydrated mycelium can be rehydrated with flavor, color, fats, or other functional food ingredients by combining dehydrated mycelium and marinade in a vacuum. A vacuum allows for greater penetration of marinade especially for thicker pieces.

In another example, dehydrated mycelium is rehydrated in a marinade having a temperature greater than 74° C. (165° F.). Not only does this provide a kill step in food manufacturing that kills any live fungal cells that may be present in the mycelium mass, but the hot marinade acts to break down mycelium cellular components and provide a more “tender” texture. The hot marinade can be performed for as little as 5 minutes or up to 24 hours depending on the desired tenderness. Another added benefit of this method is to speed up the process of penetrating marinade throughout the mycelium.

In another example, dehydrated mycelium is rehydrated in a marinade having a temperature less than 10° C. (50° F.). A cold marinade provides additional food safety for lengthier marination times. The cold marinade can be performed for as little as 5 minutes or up to 24 hours depending on the thickness of the pieces. Another added benefit of this method is to reduce any off flavors from the mycelium (i.e., reduce the contribution of any inherent flavors being produced by the mycelium).

In another example, a second dehydration step can occur following the hot marinade step. For example, this dehydration step can occur anywhere from 51° C. to 94° C. (125° F. to 200° F.) at a variety of humidity ranges. By reducing moisture following the marinade, a denser texture can be achieved, for example, having a texture similar to cooked chicken breast versus a raw chicken breast. The final moisture can also simulate textures ranging from a rare beef steak to a well-done beef steak. The final moisture content can be as low as 50% or lower if imitating a beef jerky.

In another example, molded mycelium slices are placed on a perforated tray and placed in a commercial dehydrator. Dehydration parameters are set to a temperature of 60° C. and relative humidity less than 20%. The dehydration process is stopped once the moisture content of the mycelium reaches 60%. Once rehydrated, the mycelium now has a more delicate texture than the original mycelium despite being similar moisture contents. This method can be used to imitate the texture of fish.

The rehydrated mycelium mass with added ingredients can be used in a single or combination of ways. For example, the rehydrated mycelium mass can be cooked at a temperature of less than 100° C. (e.g., 90° C., 80° C., 75° C., or 50° C., inclusive) for 1-60 minutes in dry or steam environment. The rehydrated mycelium mass can be cooked at a temperature range of 100° C. to 200° C. (e.g., 100° C., 125° C., 150° C., or 200° C., inclusive) for 1-60 minutes in dry or steam environment. The rehydrated mycelium mass can be cooked in a water bath at less than 100° C. for 1 minute to 120 minutes (e.g., 1, 2, 5, 10, 20, 40, 60, 80, 100, or 120 minutes, inclusive).

In some embodiments, the rehydrated mycelium mass can be stored. The rehydrated mycelium mass can include additional ingredients. The rehydrated mycelium mass can be cooked. The rehydrated mycelium mass can be frozen at less than 0° C. under ambient or vacuum conditions, and/or refrigerated at less than 5° C. under ambient or vacuum conditions. The rehydrated mycelium mass can be stored indefinitely in sealed container.

In some embodiments, the rehydrated mycelium mass can be packaged in a vacuum seal bag. The bag can be placed in a water bath ranging from 50° C. to 100° C., inclusive for time periods of 5 minutes to 24 hours, inclusive. The rehydrated mycelium mass is therefore pasteurized and considered a ready-to-eat food.

Producing the rehydrated mycelium mass can include tuning the texture of the rehydrated mycelium mass. Texture of the rehydrated mycelium mass can be tuned by chemical washing of the mycelium mass. Alternatively, texture can be altered by controlling the water content of the mycelium mass. Texture can also be altered through the addition of different nutrients which determine mycelium mass growth and morphology. The density of the final mycelium mass can be controlled by altering initial water content and drying conditions to produce a heavier or lighter end product.

The edible meat substitute product can include a rehydrated mycelium mass in a range of 10 wt % to 100 wt % (e.g., 10 wt %, 20 wt %, 30 wt %, 40 wt %, 50 wt %, 60 wt %, 70 wt %, 80 wt %, or 100 wt %, inclusive). The edible meat substitute product can have a water content in a range of 0 wt % to 100 wt % (e.g., 0 wt %, 10 wt %, 20 wt %, 30 wt %, 40 wt %, 50 wt %, 60 wt %, 70 wt %, 80 wt %, or 100 wt %, inclusive). In some embodiments, the fibrous mycelium mass is in a range of 10 wt % to 50 wt %, inclusive and the water content is in a range of 50 wt % to 90 wt %, inclusive. In some embodiments, the edible meat substitute product includes a soluble protein in a range of 1 wt % to 20 wt % (e.g., 1 wt %, 2 wt %, 5 wt %, 10 wt %, 15 wt %, or 20 wt %, inclusive). The edible meat substitute product can include a thickener content in a range of 0.01 wt to 5 wt % (e.g., 0.01 wt %, 0.05 wt %, 0.1 wt %, 1 wt %, 2 wt %, or 5 wt %, inclusive). The edible meat substitute product can include a fat source in a range of 0 wt % to 10 wt % (e.g., 0 wt %, 0.5 wt %, 1 wt %, 2 wt %, 5 wt %, or 10 wt %, inclusive).

The edible meat substitute product can include a flavorant. A flavorant can include flavorings or food additives. For example, the flavorant can include an oil, such as a nut-derived oil, vegetable-derived oil, plant-derived oil, and animal-derived oil. The flavorant can include spices (e.g., black pepper, fennel, mustard, nutmeg, cinnamon, ginger, cayenne pepper, clove, etc.). The flavorant can include a flavored powder (e.g., onion powder, garlic powder, BBQ powder, sour cream powder, lemon powder, lime powder, etc.).

The edible meat substitute product can include a combined methionine and cysteine content of at least 20 mg/gram crude protein. In some embodiments, the combined methionine and cysteine content in the edible meat substitute product is in a range of 20 mg/gram to 30 mg/gram (e.g., 20 mg/gram, 25 mg/gram, or 30 mg/gram, inclusive). The edible meat substitute product can have a PDCAAS score of 1. The edible meat substitute product can have an internal pH in a range of 2 to 9 (e.g., 2, 3, 4, 5, 6, 7, 8, or 9, inclusive). The edible meat substitute product can have a protein dry weight in a range of 20 wt % to 70 wt % (e.g., 20 wt %, 30 wt %, 40 wt %, 50 wt %, 60 wt %, or 70 wt %, inclusive). The edible meat substitute product can have a fiber dry weight in a range of 5 wt % to 30 wt % (e.g., 5 wt %, 10 wt %, 15 wt %, 20 wt %, 25 wt %, or 30 wt %, inclusive). The edible meat substitute product can have a dry fat weight of 0 wt % to 20 wt % (e.g., 0 wt %, 1 wt %, 5 wt %, 10 wt %, 15 wt %, or 20 wt %, inclusive). The edible meat substitute product can have a color represented by a CIE L* value of greater than 55. The edible meat substitute product can have a hardness of greater than 0.00035 kgf/mm².

The edible meat substitute product can include a chicken substitute product, a beef substitute product, a pork substitute product, a veal substitute product, or a fish substitute product. The edible meat substitute product can include 10 wt % to 90 wt % of the rehydrated mycelium mass (e.g., 10 wt %, 20 wt %, 30 wt %, 40 wt %, 50 wt %, 60 wt %, 70 wt %, 80 wt %, or 90 wt %, inclusive).

The chicken substitute product can include 50 wt % to 90 wt % water (e.g., 50 wt %, 60 wt %, 70 wt %, 80 wt %, or 90 wt %, inclusive). The chicken substitute product can include 10 wt % to 50 wt % fungal mycelium such as from N. crassa (e.g., 10 wt %, 20 wt %, 30 wt %, 40 wt %, or 50 wt %, inclusive). The chicken substitute product can include 1 wt % to 20 wt % soluble protein (e.g., 1 wt %, 2 wt %, 5 wt %, 10 wt %, or 20 wt %, inclusive). The soluble protein can include pea, egg white, and potato, among others. The chicken substitute product can include 0.01 wt % to 5 wt % thickener (e.g., 0.01 wt %, 0.05 wt %, 0.1 wt %, 1 wt %, 2 wt %, or 5 wt %, inclusive). The thickener can include pectin, carrageenan, agar, among others. The chicken substitute product can include 0 wt % to 10 wt % fat source (0 wt %, 1 wt %, 2 wt %, 3 wt %, 4 wt %, 5 wt %, or 10 wt %, inclusive). The fat source can include vegetable oils, seeds, among others. The chicken substitute product can include seasonings. The chicken substitute product can have various physical properties. For example, the chicken substitute product can have an internal pH in a range of 2 and 9 (e.g., 2, 3, 4, 5, 6, 7, 8, or 9, inclusive). The chicken substitute product can have a 20 wt % to 70 wt % protein dry weight (e.g., 20 wt %, 30 wt %, 40 wt %, 45 wt %, 50 wt %, 55 wt %, 60 wt %, 65 wt %, or 70 wt %). The chicken substitute product can have a 5 wt % to 30 wt % fiber dry weight (e.g., 5 wt %, 10 wt %, 15 wt %, 20 wt %, 25 wt %, or 30 wt %, inclusive). The chicken substitute product can have a 0 wt % to 10 wt % fat dry weight (0 wt %, 1 wt %, 2 wt %, 4 wt %, 5 wt %, or 10 wt %, inclusive). The chicken substitute product can have a CIE L* value greater than 55. The chicken substitute product can have a hardness greater than 0.00035 kgf/mm².

The meat substitute product can include 0 wt % to 90 wt % water (e.g., 0 wt %, 10 wt %, 20 wt %, 30 wt %, 40 wt %, 50 wt %, 60 wt %, 70 wt %, 80 wt %, or 90 wt %, inclusive). The meat substitute product can include 10 wt % to 100 wt % fungal mycelium such as from N. crassa (e.g., 10 wt %, 20 wt %, 30 wt %, 40 wt %, 50 wt %, 60 wt %, 70 wt %, 80 wt %, 90 wt %, or 100 wt %, inclusive). The meat substitute product can include 1 wt % to 20 wt % soluble protein (e.g., 1 wt %, 2 wt %, 5 wt %, 10 wt %, or 20 wt %, inclusive). The soluble protein can include pea, egg white, and potato, among others. The meat substitute product can include 0 wt % to 5 wt % thickener (e.g., 0 wt %, 0.01 wt %, 0.05 wt %, 0.1 wt %, 1 wt %, 2 wt %, or 5 wt %, inclusive). The thickener can include pectin, carrageenan, agar, among others. The meat substitute product can include 0 wt % to 50 wt % fat source (0 wt %, 10 wt %, 20 wt %, 30 wt %, 40 wt %, or 50 wt %, inclusive). The fat source can include vegetable oils, seeds, among others. The meat substitute product can include seasonings.

The rehydrated mycelium mass flavor can be enhanced by adding different oils. Non-limiting examples of oils include nut-derived, vegetable-derived, plant-derived, and animal-derived. Oils can be added to the food-grade residual water streams to have the multi-purpose of acting as an antifoaming agent, a carbon source for the fungus, and to integrate extra/intracellularly into the mycelium mass. Alternatively, oil can be integrated into the mycelium mass following harvesting or following cooking.

Texture of the rehydrated mycelium mass can be tuned by chemical washing of the rehydrated mycelium mass. Alternatively, texture can be altered by controlling the water content of the rehydrated mycelium mass. Texture can also be altered through the addition of different nutrients which determine rehydrated mycelium mass growth and morphology. The density of final rehydrated mycelium mass can be controlled by altering initial water content and drying conditions to produce a heavier or lighter end product.

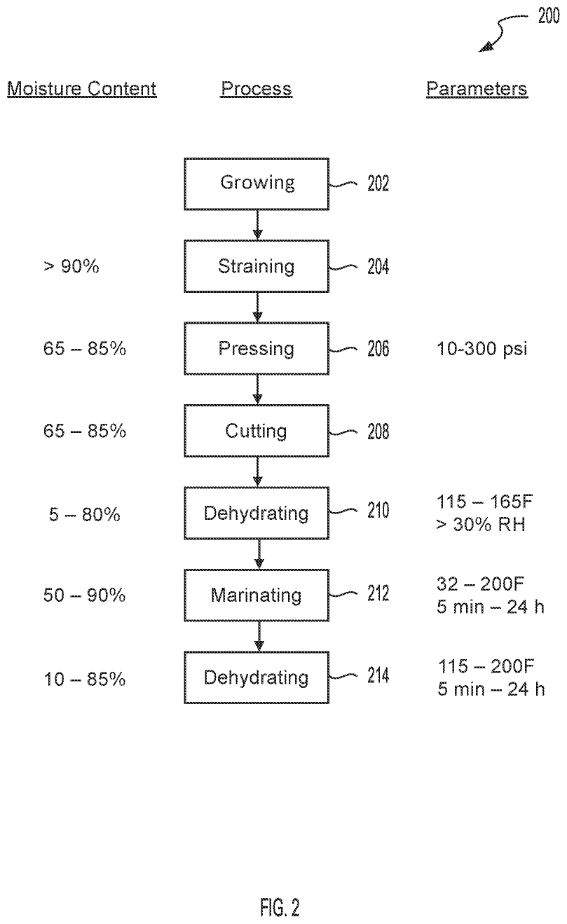

FIG. 2 illustrates a flow chart of an example method for dehydrating and rehydrating mycelium to obtain an edible meat substitute, according to an embodiment. The method 200 may include a growing step, at 202. The method may include a straining step, at 204. The method may include a pressing step, at 206. The method may include a cutting step, at 208. The method may include a first dehydrating step, at 210. The method may include a marinating step, at 212. The method may include a second dehydrating step, at 214.

In further detail, the method 200 may include a growing step, at 202. For example, the method 200 may include growing fungal cells in a growth media. The growth media may be contained in a vessel, such as a vat capable of growing several kilograms of the fungal mycelium. The growth media can be referred to as an original growth media. The method 200 may include growing fungal cells in a growth media such that the fungal cells produce mycelium. The growth media can include nutrients (e.g., sugar, nitrogen-containing compounds, or phosphate-containing compounds). The growth media can include a sugar, a nitrogen-containing compound, and a phosphate-containing compound. The sugar can be in the range of 5 g/L to 50 g/L. For example, the sugar can be 5 g/L, 10 g/L, 20 g/L, 30 g/L, 40 g/L, or 50 g/L, inclusive. The sugar can include sucrose, glucose, fructose, molasses, or a mixture of sugars. The nitrogen-containing compound can be in the range of 0.5 g/L to 10 g/L. For example, the nitrogen-containing compound can be 0.5 g/L, 1 g/L, 2 g/L, 3 g/L, 4 g/L, 5 g/L, or 10 g/L, inclusive. The nitrogen-containing compound can include ammonium hydroxide, ammonium nitrate, ammonium sulfate, ammonium chloride, urea, yeast extract, peptone, or a mixture of nitrogen-containing compounds. The phosphate-containing compound can be in the range of 0.1 g/L to 5 g/L. For example, the phosphate-containing compound can be 0.1 g/L, 0.2 g/L, 0.3 g/L, 0.4 g/L, 0.5 g/L, 1 g/L, 2 g/L, 3 g/L, 4 g/L, or 5 g/L, inclusive. The phosphate-containing compound can be potassium phosphate, sodium phosphate, phosphoric acid, or a mixture of phosphate-containing compounds.

The method 200 may include a straining step, at 204. For example, the straining the mycelium can include includes separating the mycelium mass from the growth media. Separating the mycelium mass from the growth media can be performed using gravity straining, centrifugation, a belt press, a filter press, a mechanical press, a drum dryer, or any other suitable process. The separated mycelium mass can have a moisture content of greater than 90 wt %. For example, the separated mycelium mass can have a moisture content of 91 wt %, 92 wt %, 93 wt %, 94 wt %, 95 wt %, 96 wt %, 97 wt %, 98 wt %, or 99 wt %, inclusive. During the separation process, the mycelium mass can be washed with water, ethanol, acid, base or other solvent. Recovered filtrate can be reused or discarded. Cell walls of the mycelium mass can be disrupted, for example, through lysing. Lysis may be performed by adjusting the pH to below 4 or above 9, by adding lysis enzymes, by raising the temperature in a range of 40° C. and 60° C. in a range of 1 and 24 hours, or any other suitable lysis method. Following separation, additives (e.g., food additives) can be mixed with the mycelium mass. Additives can include vegetable or animal proteins, fats, emulsifiers, thickeners, stabilizers, and flavoring, for example, when the mycelium mass is being formed into an edible product. The moisture content of the mycelium mass during the straining step can be greater than 90 wt %.

The method 200 may include a pressing step, at 206. Pressing the mycelium mass may include pressing the mycelium mass to produce a compacted mycelium mass. The compacted mycelium mass can be referred to as a pressed mycelium mass. The compacted mycelium mass can have a moisture content in a range of 65 wt % to 85 wt %. For example, the compacted mycelium mass can have a moisture content of 65 wt %, 70 wt %, 75 wt %, 80 wt %, or 85 wt %, inclusive. A pressure in a range of 10 psi and 300 psi can be applied to the mycelium mass to produce a compacted mycelium mass. For example, a pressure of 10 psi, 25 psi, 50 psi, 75 psi, 100 psi, 125 psi, 150 psi, 175 psi, 200 psi, 225 psi, 250 psi, 275 psi, or 300 psi can be applied to the mycelium mass.

The method 200 may include a cutting step, at 208. The compacted mycelium mass can be cut into pieces of various shapes and sizes. For example, the compacted mycelium mass can be sliced, cut, or broken up into pieces. The pieces of compacted mycelium mass can have a moisture content in a range of 65 wt % to 85 wt %, inclusive.

The method 200 may include a first dehydrating step, at 210. The pieces of compacted mycelium mass can be dehydrated at a temperature in a range of 40° C. to 80° C. and a relative humidity of greater than 30%. For example, the pieces of compacted mycelium mass can be dehydrated at a temperature of 40° C., 45° C., 50° C., 55° C., 60° C., 65° C., 70° C., 75° C., or 80° C., inclusive. The pieces of dehydrated mycelium mass can have a moisture content in a range of 5 wt % to 80 wt %. For example, the pieces of dehydrated mycelium mass can have a moisture content of 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 75 wt %, or 80 wt %, inclusive.

The method 200 may include a marinating step, at 212. The pieces of dehydrated mycelium mass can be marinated at a temperature in a range of 0° C. and 100° C. for 5 minutes to 24 hours. The pieces of rehydrated mycelium mass can have a moisture content in a range of 50 wt % to 90 wt %. For example, the pieces of rehydrated mycelium mass can have a moisture content of 50 wt %, 55 wt %, 60 wt %, 65 wt %, 75 wt %, 80 wt %, 85 wt %, or 90 wt %, inclusive. The piece of dehydrated mycelium mass can be rehydrated at a temperature of 0° C., 5° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., or 100° C., inclusive. The piece of dehydrated mycelium mass can be rehydrated for 5 minutes, 30 minutes, 1 hour, 2 hours, 5 hours, 10 hours, 15 hours, 20 hours, or 24 hours, inclusive.

The method 200 may include a second dehydrating step, at 214. The pieces of rehydrated mycelium mass can be dehydrated at a temperature in a range of 40° C. and 100° C., inclusive, for 5 minutes to 24 hours, inclusive. The pieces of dehydrated mycelium mass can have a moisture content in a range of 10 wt % to 85 wt %. For example, the piece of dehydrated mycelium mass can have a moisture content of 10 wt %, 15 wt %, 20 wt %, 25 wt %, 30 wt %, 35 wt %, 40 wt %, 45 wt %, 50 wt %, 55 wt %, 60 wt %, 65 wt %, 75 wt %, 80 wt %, or 85 wt %, inclusive. The piece of rehydrated mycelium mass can be dehydrated at a temperature of 40° C., 45° C., 50° C., 55° C., 60° C., 65° C., 70° C., 75° C., 80° C., 85° C., 90° C., 95° C., or 100° C., inclusive. The piece of rehydrated mycelium mass can be dehydrated for 5 minutes, 30 minutes, 1 hour, 2 hours, 5 hours, 10 hours, 15 hours, 20 hours, or 24 hours, inclusive.

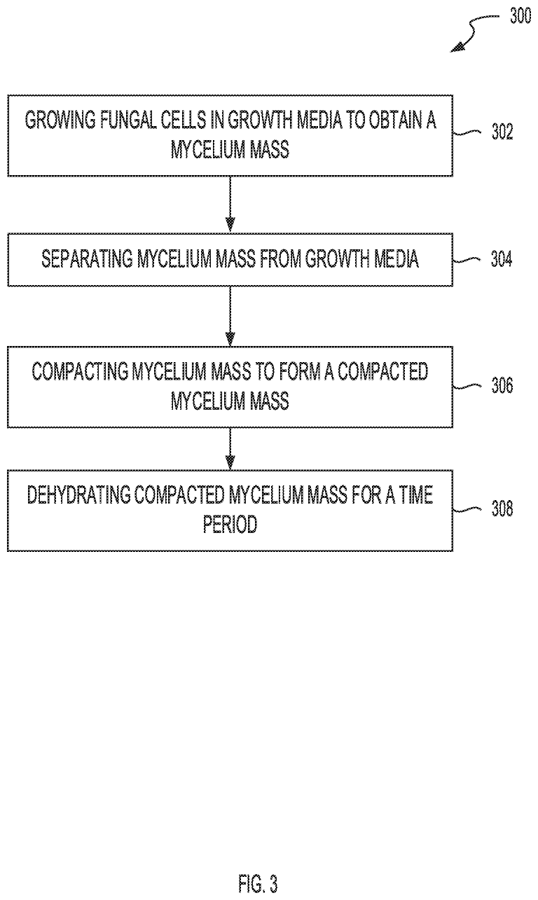

FIG. 3 illustrates a flow chart of an example method for dehydrating mycelium, according to an embodiment. In brief overview, the method 300 may include growing fungal cells in a growth media, at 302. The method 300 may include separating mycelium mass from the growth media, at 304. The method 300 may include compacting the mycelium mass, at 306. The method 300 may include dehydrating the compacted mycelium mass, at 308.

In further detail, the method 300 may include growing fungal cells in a growth media, at 302. For example, the growth media may be contained in a vessel, such as a vat capable of growing several kilograms of the fungal mycelium. The growth media can be referred to as an original growth media. The method 300 may include growing fungal cells in a growth media such that the fungal cells produce mycelium. The growth media can include nutrients (e.g., sugar, nitrogen-containing compounds, or phosphate-containing compounds). The growth media can include a sugar, a nitrogen-containing compound, and a phosphate-containing compound. The sugar can be in the range of 5 g/L to 50 g/L. For example, the sugar can be 5 g/L, 10 g/L, 20 g/L, 30 g/L, 40 g/L, or 50 g/L, inclusive. The sugar can include sucrose, glucose, fructose, molasses, or a mixture of sugars. The nitrogen-containing compound can be in the range of 0.5 g/L to 10 g/L. For example, the nitrogen-containing compound can be 0.5 g/L, 1 g/L, 2 g/L, 3 g/L, 4 g/L, 5 g/L, or 10 g/L, inclusive. The nitrogen-containing compound can include ammonium hydroxide, ammonium nitrate, ammonium sulfate, ammonium chloride, urea, yeast extract, peptone, or a mixture of nitrogen-containing compounds. The phosphate-containing compound can be in the range of 0.1 g/L to 5 g/L. For example, the phosphate-containing compound can be 0.1 g/L, 0.2 g/L, 0.3 g/L, 0.4 g/L, 0.5 g/L, 1 g/L, 2 g/L, 3 g/L, 4 g/L, or 5 g/L, inclusive. The phosphate-containing compound can be potassium phosphate, sodium phosphate, phosphoric acid, or a mixture of phosphate-containing compounds.

The fungal cells can be grown at a temperature in a range of 25° C. to 40° C., inclusive. The fungal cells can be grown in a range of 12 hours to 48 hours, inclusive. Growing fungal cells can produce a yield of 5 g/L to 20 g/L of fungal cell dry weight. The mycelium can have a protein content of greater than 40 wt % (dry weight). In some embodiments, the mycelium may have a protein content of 50% to 65%, inclusive (dry weight). The mycelium can have a combined methionine and cysteine content of at least 25 mg/g crude protein.

In some embodiments, the method 300 may include removing a volume of a broth. The broth can contain the fungal cells and the growth media. Removing a volume of broth can include discretely removing a volume of broth. For example, a volume of broth can be siphoned from a container containing the broth in a batch process, or be continuously removed from the broth. For example, a volume of broth can flow out of the container containing the broth in a continuous process.

The method 300 may include adding fresh growth media to a container containing the broth. The broth can be a fermentation broth. Nutrients (e.g., sugar, phosphate-containing compound, or nitrogen-containing compound) can be added in a batch growth configuration. For example, the nutrients can be added after a predetermined amount of time (e.g., after 1 hour, 2 hours, 3 hours, 6 hours, or 12 hours). The concentrations of none or at least one of the nutrients of the fresh growth media can be brought to the concentrations of nutrients of the original growth media described in operation 302. The fresh growth media can have a volume that is greater than, less than, or equal to a volume of growth media that was lost from the original growth media during growth of the fungal cells in the original growth media.

In one example, after 6 hours, the concentration of sugar, phosphate-containing compound, and nitrogen-containing compound in the fresh growth media is increased. Nutrients are added to bring the concentrations of sugar, phosphate-containing compound, and nitrogen-containing compound of the broth to the concentrations of sugar, phosphate-containing compound, and nitrogen-containing compound, respectively of the original growth media.

In one example, after at least 12 hours, 50% to 95% of the broth can be removed. Fresh media can be added containing nutrients (e.g., sugar, phosphate-containing compound, or nitrogen-containing compound). The nutrient concentration of the broth can be increased by added fresh growth media.

Nutrients can be added in a continuous growth configuration. For example, a volume of broth (e.g., 0.01 vol %, 1 vol %, 5 vol %, 10 vol %, 25 vol %, 50 vol %, or 95 vol %, inclusive) can be removed from the container containing the fungal cells and the growth media. Fresh growth media can be added to the container containing the broth. The fresh growth media can be provided as a continuous flow. The volume of the broth in the container can be monitored to stay at a specified level. For example, the volume of the broth in the container can stay at a fixed volume. The volume of fresh growth media that is added can be equal to the volume of broth that is lost from the container.

The method 300 may include growing fungal cells in a growth media such that the fungal cells produce a mycelium mass having a protein content of greater than 40 wt % of a dry mass of the mycelium mass. For example, the mycelium mass can have a protein content of 45 wt %, 50 wt %, 60 wt %, 70 wt %, 80 wt %, or 90 wt %, inclusive, of the dry mass of the mycelium mass.

The method 300 includes separating the mycelium mass from the growth media, at 304. Separating the mycelium mass from the growth media can be performed using gravity straining, centrifugation, a belt press, a filter press, a mechanical press, a drum dryer, or any other suitable process. The separated mycelium mass can have a moisture content of greater than 90 wt %. For example, the separated mycelium mass can have a moisture content of 91 wt %, 92 wt %, 93 wt %, 94 wt %, 95 wt %, 96 wt %, 97 wt %, 98 wt %, or 99 wt %, inclusive. During the separation process, the mycelium mass can be washed with water, ethanol, acid, base or other solvent. Recovered filtrate can be reused or discarded. Cell walls of the mycelium mass can be disrupted, for example, through lysing. Lysis may be performed by adjusting the pH to below 4 or above 9, by adding lysis enzymes, by raising the temperature in a range of 40° C. and 60° C. in a range of 1 and 24 hours, or any other suitable lysis method. Following separation, additives (e.g., food additives) can be mixed with the mycelium mass. Additives can include vegetable or animal proteins, fats, emulsifiers, thickeners, stabilizers, and flavoring, for example, when the mycelium mass is being formed into an edible product.

The method 300 may include compacting the mycelium mass, at 306. Compacting the mycelium mass may include compacting the mycelium mass to produce a compacted mycelium mass. The compacted mycelium mass can have a moisture content in a range of 65 wt % to 85 wt %. For example, the compacted mycelium mass can have a moisture content of 65 wt %, 70 wt %, 75 wt %, 80 wt %, or 85 wt %, inclusive.

The method 300 may include dehydrating the compacted mycelium mass, at 308. Dehydrating the compacted mycelium mass may include dehydrating the compacted mycelium mass to produce a dehydrated mycelium mass. The dehydrated mycelium mass can have a moisture content of less than 20 wt %. For example, the dehydrated mycelium mass can have a moisture content of 5 wt %, 10 wt %, 15 wt %, or 19 wt %, inclusive. Dehydrating the compacted mycelium mass can include dewatering the mycelium mass. For example, the compacted mycelium mass can be thermally dried, for example, to a moisture content of less than 20 wt %. The compacted mycelium mass can be thermally dried at a specified temperature. For example, the compacted mycelium mass can be thermally dried at a temperature of 135° C. The compacted mycelium mass can be thermally dried at a temperature in a range of 30° C. to 60° C. The compacted mycelium mass can be thermally dried at a relative humidity of greater than 30%. For example, the compacted mycelium mass can be thermally dried at a relative humidity of 35%, 40%, 45%, or 50%, inclusive. In some embodiments, the compacted mycelium mass can be thermally dried with forced air. In some embodiments, dewatering the compacted mycelium mass can include applying a mechanical force (e.g., a press, a sieve) to remove water from the mycelium. Dehydrating (e.g., via thermal or mechanical drying) the compacted mycelium mass can produce a dehydrated mycelium mass. The dehydrated mycelium mass can be ground to reduce particle size, for example, to create a powder.

The compacted mycelium mass can be dehydrated for a time period to produce a dehydrated mycelium mass having a moisture content of less than 20 wt %. For example, the compacted mycelium mass can be dehydrated for a time period of greater than 8 hours (e.g., 10 hours, 15 hours, 20 hours, or 24 hours, etc.). The dehydrated mycelium mass can have a volume that is less than 50% of the compacted mycelium mass. For example, the dehydrated mycelium mass can have a volume that is 10%, 20%, 30%, 40%, or 50%, inclusive, of the compacted mycelium mass.

Following are some examples of growing fungi and dehydrating a mycelium mass. These examples are for illustrative purposes only and should not be construed as limiting the disclosure in any shape or form.

In one example, Neurospora crassa (N. crassa) was grown in a 200 L stirred tank bioreactor under pre-set conditions. Following full growth, N. crassa biomass (mycelium) was collected through a 200 PEM nylon mesh bag. The moisture content of the mycelium at this stage was approximately 95%. The high moisture mycelium is added to a perforated, stainless-steel mold consisting of base and follower. A pressure of 100 psi is applied to the mold lid compacting the mycelium into a rigid block of approximately 75% moisture content. The block is then sliced or broken into pieces of desired size and shape. Molded mycelium slices are then placed onto a perforated tray and placed in a dehydrator. The dehydration parameters are set to a temperature of 60° C. and relative humidity greater than 30%. The slices become very dense during dehydration and take in excess of 8 hours to achieve less than 20% moisture content. The final volume of the dehydrated mycelium can be less than 50% of the compacted mycelium mass.

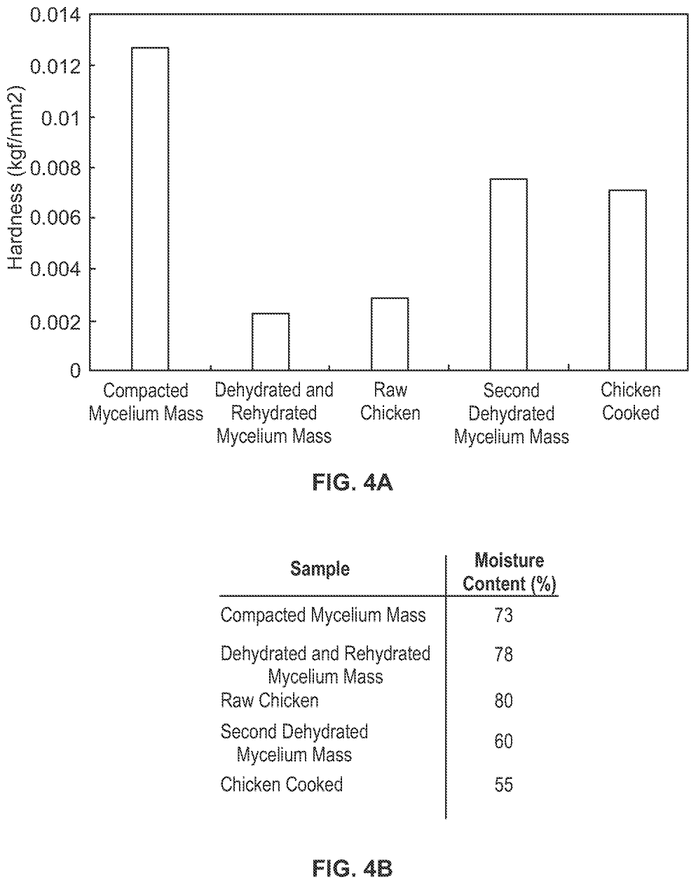

FIG. 4A illustrates a bar chart of hardnesses of a compacted mycelium mass, a dehydrated and rehydrated mycelium mass, a second dehydrated mycelium mass produced by dehydrating the rehydrated mycelium mass, raw chicken, and cooked chicken. The compacted mycelium mass can have a hardness of between 0.012 kgf/mm² and 0.014 kgf/mm². The dehydrated and rehydrated mycelium mass can have a hardness of between 0.0018 kgf/mm² and 0.0035 kgf/mm². The second dehydrated mycelium mass can have a hardness of between 0.007 kgf/mm² and 0.0088 kgf/mm². The raw chicken can have a hardness of between 0.0018 kgf/mm² and 0.0035 kgf/mm², which is in the same range as the dehydrated and rehydrated mycelium mass. The cooked chicken can have a hardness of between 0.007 kgf/mm² and 0.0088 kgf/mm², which is in the same range as the second dehydrated mycelium mass.

FIG. 4B illustrates a table of moisture contents of a compacted mycelium mass, a dehydrated and rehydrated mycelium mass, a second dehydrated mycelium mass produced by dehydrating the rehydrated mycelium mass, raw chicken, and cooked chicken. The compacted mycelium mass can have a moisture content of 73%. The dehydrated and rehydrated mycelium mass can have a moisture content of 78%. The second dehydrated mycelium mass can have a moisture content of 60%. The raw chicken can have a moisture content of 80%, which is similar to the moisture content of the dehydrated and rehydrated mycelium mass. The cooked chicken can have a moisture content of 55%, which is similar to the moisture content of the second dehydrated mycelium mass. Therefore, dehydrated and rehydrated mycelium mass can mimic the hardness and moisture content of raw chicken, and the second dehydrated mycelium mass can mimic the hardness and moisture content of cooked chicken, thereby providing a consumer of a such a dehydrated and rehydrated and dehydrated mycelium mass, a very similar experience to eating chicken.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

As used herein, the singular forms “a”, “an” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

Reference throughout this specification to “one embodiment,” “an embodiment,” or similar language means that a particular feature, structure, or characteristic described in connection with the embodiment is included in at least one embodiment of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the present disclosure, however, absent an express correlation to indicate otherwise, an implementation may be associated with one or more embodiments.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A method comprising:
growing fungal cells in a growth media such that the fungal cells produce a mycelium mass having a protein content of greater than 40 wt % of a dry mass of the mycelium mass;
separating the mycelium mass from the growth media;
compacting the mycelium mass to produce a compacted mycelium mass having a moisture content in a range of 65 wt % to 85 wt %;
dehydrating the compacted mycelium mass to produce a dehydrated mycelium mass having a moisture content in a range of 5 wt % to 60 wt % and a first hardness in a range of 0.007 kgf/mm² to 0.018 kgf/mm²; and
rehydrating the dehydrated mycelium mass to form a rehydrated mycelium mass having a moisture content of greater than 60 wt % and a second hardness in a range of 0.00035 kgf/mm² to 0.007 kgf/mm².

2. The method of claim 1, wherein the dehydrated mycelium mass has a moisture content in a range of 5 wt % to 20 wt %.

3. The method of claim 1, wherein a volume of the dehydrated mycelium mass is equal to or greater than 75% of a volume of the compacted mycelium mass.

4. The method of claim 1, wherein the compacted mycelium mass is dehydrated to form the dehydrated mycelium mass at a temperature of less than 60° C., and a humidity of less than 30%.

5. The method of claim 1, further comprising:
dehydrating the rehydrated mycelium mass to produce a second dehydrated mycelium mass having a moisture content in a range of 45 wt % to 75 wt % and a third hardness in a range of 0.0035 kgf/mm² to 0.014 kgf/mm²;
wherein the rehydrated mycelium mass is dehydrated at a temperature in a range of 40° C. to 100° C. and a relative humidity of less than 20%.

6. The method of claim 1, further comprising:
dehydrating the rehydrated mycelium mass to produce a second dehydrated mycelium mass having a moisture content of less than 50 wt % and a third hardness in a range of 0.011 kgf/mm² to 0.042 kgf/mm²;
wherein the rehydrated mycelium mass is dehydrated at a temperature in a range of 40° C. to 100 degrees Celsius and a relative humidity of less than 20%.

7. The method of claim 1, wherein rehydrating the dehydrated mycelium mass comprises soaking the dehydrated mycelium mass in an aqueous solution comprising a flavor, a food coloring, fats, spices, and/or additives.

8. The method of claim 1, wherein rehydrating the dehydrated mycelium mass comprises soaking the dehydrated mycelium mass for a time period in a marinade heated to a temperature sufficient to kill greater than 99% of live fungal cells present in the dehydrated mycelium mass.

9. The method of claim 1, wherein separating the mycelium mass from the growth media produces a separated mycelium mass, the separated mycelium mass having a moisture content of greater than 90 wt %.

10. The method of claim 1, wherein a volume of the dehydrated mycelium mass is equal to or greater than 85% of a volume of the compacted mycelium mass.

11. The method of claim 1, wherein a volume of the dehydrated mycelium mass is equal to or greater than 95% of a volume of the compacted mycelium mass.

12. A method, comprising:
growing fungal cells in a growth media such that the fungal cells produce a mycelium mass having a protein content of greater than 40 wt % of a dry mass of the mycelium mass;
separating the mycelium mass from the growth media;
compacting the mycelium mass to produce a compacted mycelium mass having a moisture content in a range of 65 wt % to 85 wt %; and
dehydrating the compacted mycelium mass at a temperature in a range of 30° C. to 60° C. and a relative humidity in a range of 30% to 50% for a time period to produce a dehydrated mycelium mass having a moisture content of less than 20 wt %, the dehydrated mycelium mass having a volume that is less than 50% of the compacted mycelium mass.

13. The method of claim 12 wherein the time period is greater than 8 hours.

14. The method of claim 12, wherein separating the mycelium mass from the growth media produces a separated mycelium mass having a moisture content of greater than 90 wt %.

15. The method of claim 12, wherein the dehydrated mycelium mass has a moisture content in a range of 5 wt % to 20 wt %.

16. The method of claim 12, wherein a volume of the dehydrated mycelium mass is equal to or greater than 75% of a volume of the compacted mycelium mass.

17. The method of claim 12, wherein a volume of the dehydrated mycelium mass is equal to or greater than 85% of a volume of the compacted mycelium mass.

18. The method of claim 12, wherein a volume of the dehydrated mycelium mass is equal to or greater than 95% of a volume of the compacted mycelium mass.

19. The method of claim 12, wherein separating the mycelium mass from the growth media produces a separated mycelium mass, the separated mycelium mass having a moisture content of greater than 95 wt %.

20. The method of claim 12, further comprising:
providing an edible meat substitute product comprising the dehydrated mycelium mass.
